(19) United States
(12) Patent Application Publication
(10) Pub. No.: US 2024/0239737 A1
(43) Pub. Date: Jul. 18, 2024

(54) Synthesis of Triisoamyl Citrate (Triisopentanol) and its Applications as a Solvent, Plastizer and Component of Cosmetic and Aerosol Formulations

(71) Applicant: BRASIL BIO FUELS S.A, SÃO JOÃO DA BALIZA (BR)

(72) Inventor: Setsuo Sato, Piracicaba (BR)

(21) Appl. No.: 18/413,539

(22) Filed: Jan. 16, 2024

(30) Foreign Application Priority Data
Jan. 13, 2023 (BR) 10 2023 000746 5

(51) Int. Cl. C07C 67/56; C07C 69/34

(52) U.S. Cl. CPC C07C 67/56; C07C 69/34

(57) ABSTRACT

The present invention is based on the synthesis of triisoamyl citrate (triisopentanol citrate) and its applications as a plasticizer for polyvinyl chloride polymers, natural and synthetic rubbers, industrial solvents, replacing petrochemical-based products such as Phthalates (Dioctyl Phthalate, Diisononyl Phthalate, Dibutyl Phthalate), Acetylated tributylcitrate, epoxidized fatty esters, isoparaffins. It is a 100% renewable, high-performance, non-toxic, biodegradable product with an extremely competitive cost. With the increase in corn alcohol production in Brazil, this technology and product adds value to the fusel oil co-product generated in corn alcohol plants.

### CROSS REFERENCE TO RELATED APPLICATION

The present invention claims the benefit of priority to Brazilian Patent Application No. 10 2023 000746 5 filed on Jan. 13, 2023 with the Brazilian Patent Office, the entire contents of which are incorporated herein by reference in its entirety.

### TECHNICAL FIELD

The invention is based on the production of green plasticizers, solvents, completely biodegradable from fusel oil components originating in the production of ethanol from corn and sugar cane. Production is made using citric acid from fermentation processes. This molecule is completely free of phthalates and is mainly used in the PVC (polyvinyl chloride) segment for the production of food films and articles for the production of shoes, bags, vehicle coverings and sofas. It is also used as a solvent in agricultural formulations, in aerosols, and in cleaning formulations for the electronics industry.

The term fusel oil (Finkel) originated in Germany and referred to lower or bad fractions. Nowadays, the term fusel oil is widely used to designate the mixture of higher alcohols obtained in various phases of ethanol purification. Isoamyl and isobutyl alcohols are the main components, the isoamyl alcohol being predominant in the form of some isomers.

The composition and concentration of fusel oil ranges depending on the conditions and raw materials used.

TABLE 1
below shows some important data.

| | % V/V OF |
|---|---|
| SUBSTRATE | PRODUCED ALCOHOL |
| Sugar cane molasses | 0.1 to 0.5 |
| Evaporated sugarcane juice | 0.1 to 0.2 |
| Maize malt | 0.25 to 0.3 |
| Maize | 0.4 to 0.5 |
| Wheat | 0.2 to 0.3 |
| Potatoes | 0.5 to 1.1 |

TABLE 2
shows the typical composition of commercial fusel oil.

| Alcohol | Concentration (%) |
|---|---|
| Isoamyl | 49.6 |
| Isobutyl | 20.1 |
| Ethyl | 3.6 |
| Methyl | 2.11 |
| n-Propyl | 1.5 |

The fusel oil generated to be sold has in its composition water, ethanol, isoamyl alcohol, isobutyl alcohol and a series of other components.

The most important component for the chemical market is isoamyl alcohol, which is used for the production of esters that are used in industry as flavorings, industrial solvents, plasticizers.

Normally, isoamyl and isobutyl alcohol are purified in distillation columns, obtaining pure substances free of other components.

An application in the use of herbicides is described in the work carried out at Centro de Cana IAC, Ribeirão Preto, with the objective of evaluating the effectiveness of fusel oil alone and in mixture with glyphosate, applied in the late post-emergence of weeds of a natural community. The experimental design was in randomized blocks (DBC), with 13 treatments in four replications, with 52 plots of 3×3 m each. The treatments were applied on Mar. 22, 2006, with pressurized backpack equipment equipped comprising a bar with four Teejet 110.02 TT spray tips, adjusted to a spray volume of 212 L ha⁻¹. The percentage of control was evaluated at 14, 21, 28, 35 and 42 days after application of the treatment (DAA) and the dry mass at 42 DAA. For most treatments, there was not 100% control due to the presence of weeds of the genera Commelina and Cyperus spp., which, even with yellowed parts, were more tolerant to the application of the products. The control obtained with fusel oil applied alone did not exceed 20%, at 42 DAA.

### Citric Acid

Also known as hydrogen citrate, citric acid is a weak organic acid, having the molecular formula C₆H₈O₇, found in a solid state at room temperature, white or translucent in color, odorless, with a sour taste, completely soluble in water, biodegradable, low melting point, non-toxic, non-flammable, present in citrus compounds, such as lemon, orange, tangerine, citron, bergamot and grapefruit.

This acid is isolated from citrus fruits and obtained by the sucrose fermentation process carried out by the fungus Aspergillus niger or yeast Candida lipolytica, using sugar cane molasses or dextrose as raw material. This process gives rise to liquid citric acid, which is subsequently purified, assuming solid form. Its acidity content is due to the presence of 3 carboxyls (COOH) in the carbon chain, therefore being classified as a tricarboxylic acid.

It is the most used acid by the food and beverage industry, as it has antioxidant, acidifying, flavoring, sequestering and acidity regulating properties. In dairy products, for example, it acts as a stabilizer; helps maintain the ideal pH of sweets; in the pharmaceutical industry, it is applied to the production of anticoagulant (for blood transfusions) and effervescent medications; enhances the flavor of soft drinks and “steals” the metallic ions that change the color of the liquid (especially iron, the main element responsible for cloudiness); acts as a preservative, combating the development of microorganisms; when preparing fish and seafood, it is used to combat the appearance of stains, smells and undue flavors; it is added to the meat brine to speed curing and maintain color. In general, it preserves the flavor of industrialized drinks and foods, regulating the pH, masking the unpleasant taste of some compounds, neutralizing the sweet palate and acidifying the flavor.

Citric acid is the most important organic acid produced and used as an acidulant or pH control agent in the food, beverage, and pharmaceutical industries. Citric acid has high economic qualities due to its numerous applications in several sectors. Citric acid is available as a colorless crystal, white crystalline powder, or as a liquid solution.

The most commercially used and economically viable, citric acid is produced by the fermentation of carbohydrates found in starches and sugars. In fermentation, sugar or molasses is converted to citric acid by microbial activity with the addition of nutrients. After fermentation, the citric acid needs to be recovered and purified from the juice.

### BACKGROUND OF THE INVENTION

Patent CN105646226A reports the production of acetylated tributyl citrate (TBCA), a well-known product used on the market as a food grade plasticizer similar to CN101402571A which deals with the same subject.

Patents about the triisoamyl citrate were not found in the prior art due to the difficulty of finding pure isoamyl alcohol at competitive prices.

Patent US20090149586A1 describes the use of epoxidized isoamyl alcohol fatty esters as a plasticizer for PVC.

The objective of the invention is to provide the synthesis of triisoamyl citrate (triisopentanol citrate), a molecule chemically different from TBCA that uses a product of renewable origin and a simpler production process because it does not have the acetylation step. This also means a more commercially competitive product.

The molecule obtained allows, for example, its use as a plasticizer for PVC films for food use, articles for hospital uses such as blood transfusion tubes, for the production of synthetic leather used in the manufacture of bags, sofas, car seats, for the production of shoes, tennis soles, refrigerator boots, paints and varnishes and also nail polish formulations for nail applications.

In addition to acting as a plasticizer, the molecule object of the present invention can be used, for example, as a solvent for agrochemicals, non-toxic solvent for use in aerosol components and as a cleaning solvent for electronic circuits and other components.

### DESCRIPTION OF THE INVENTION

For the synthesis of triisoamyl citrate (triisopentanol citrate) we have the following forms.

### Example 1

In a 2000 ml reactor, load 192 grams of citric acid, 400 grams of isoamyl alcohol and 1.5 grams of methane sulfonic acid.

Under stirring, heat the product to 140° C. under reflux, separating the reaction water.

After 4 hours at a temperature of 130° ° C. to 140° ° C., 50 grams of water were collected.

The system was placed for distillation, raising the temperature to 160° C. and finally applying a vacuum of 20 mmHg to remove volatiles and odoriferous substances. At this stage, 120 grams of recovered isoamyl alcohol were collected.

The product was cooled to 80° ° C. and filtered through a buckner funnel and blue band filter paper.

TABLE 3
shows the analysis of the result.

| | |
|---|---|
| Aspect | Clear yellowish liquid |
| Acidity level | 0.4 mgkoh/gr |
| saponification index | 412 mgkoh/gr |
| Moisture | 980 ppm |

Comparative application test of triisoamyl citrate (triisopentanol citrate) obtained in example 1 with acetylated tributylcitrate (TBCA).

TABLE 4
shows the preparation of the mixture with PVC.

| Material | Plastisol 1 | Plastisol 2 |
|---|---|---|
| SP 1000 Resin | 100 | 100 |
| Triisoamylcitrate (TIIC) | 60 | 0 |
| Tributylcitrateacetylated (TBCA) | 0 | 60 |
| CA/Zn stabilizer | 1.5 | 1.5 |
| Lubricant | 0.25 | 0.25 |

To prepare the test specimens, the above mixtures were mixed in metal cups with mechanical stirring for 5 minutes, calendered and pressed in a hydraulic press.

TABLE 5
shows the results of the test specimens.

| PLASTISSOL | 1 | 2 |
|---|---|---|
| Shore A hardness | 78 | 76 |
| Traction (kgf/mm²) | 1,589 | 1,661 |
| Elongation at break (%) | 355 | 348 |
| Mass loss in 60° C. oven after 7 days (%) | 0.002 | 0.002 |
| Volatiles at 150° C. for 5 minutes (%) | 0.4 | 0.3 |

### Example 2

In a 2000 ml reactor, load 192 grams of citric acid, 600 grams of isoamyl alcohol and 2.0 grams of paratoluene sulfonic acid.

Under stirring, heat the product to 130° C. under reflux, separating the reaction water.

After 4 hours at a temperature of 120° ° C. to 130° C., 52 grams of water were collected.

The system was placed for distillation, raising the temperature to 160° C. and finally applying a vacuum of 20 mmHg to remove volatiles and odoriferous substances. At this stage, 310 grams of recovered isoamyl alcohol were collected.

The product was cooled to 80° C. and filtered through a buckner funnel and blue band filter paper.

TABLE 6
shows the analysis of the result.

| | |
|---|---|
| Aspect | clear yellowish liquid |
| Acidity level | 0.3 mgkoh/gr |
| saponification index | 408 mgkoh/gr |
| Moisture | 1200 ppm |

Comparative application test of triisoamyl citrate (triisopentanol citrate) obtained in example 2 with acetylated tributylcitrate (TBCA).

TABLE 7
shows the repair of the mixture with PVC.

| Material | Plastisol 1 | Plastisol 2 |
|---|---|---|
| SP 1000 Resin | 100 | 100 |
| Triisoamylcitrate (TIIC) | 60 | 0 |
| Tributylcitrateacetylated (TBCA) | 0 | 60 |
| CA/Zn stabilizer | 1.5 | 1.5 |
| lubricant | 0.25 | 0.25 |

To prepare the test specimens, the above mixtures were mixed in metal cups with mechanical stirring for 5 minutes, calendered and pressed in a hydraulic press.

TABLE 8
shows the results of the test specimens.

| PLASTISSOL | 1 | 3 |
|---|---|---|
| Shore A hardness | 76 | 74 |
| Traction (kgf/mm²) | 1,592 | 1,668 |
| Elongation at break (%) | 352 | 344 |
| Mass loss in 60° C. oven after 7 days (%) | 0.003 | 0.002 |
| Volatiles at 150° C. for 5 minutes (%) | 0.3 | 0.4 |

### Example 3

In a 2000 ml reactor, load 192 grams of citric acid, 920 grams of isoamyl alcohol and 2.0 grams of paratoluene sulfonic acid.

Under stirring, heat the product to 130° C. under reflux, separating the water from the fusel oil plus the reaction water.

After 4 hours at a temperature of 120° ° C. to 130° C., 372 grams of water were collected.

The system was placed for distillation, raising the temperature to 160° C., and finally applying a vacuum of 20 mmHg to remove volatiles and odoriferous substances. At this stage, 280 grams of recovered isoamyl alcohol were collected.

The product was cooled to 80° C., 10 grams of active charcoal was added, kept under stirring for 30 minutes and filtered through a buckner funnel and blue band filter paper.

TABLE 9
shows the result analysis.

| | |
|---|---|
| Aspect | clear, slightly yellowish liquid |
| Acidity level | 0.4 mgkoh/gr |
| Saponification index | 414 mgkoh/gr |
| Moisture | 1600 ppm |

Comparative application test of triisoamyl citrate (triisopentanol citrate) obtained in example 2 with acetylated tributylcitrate (TBCA).

TABLE 10
shows the preparation of the mixture with PVC.

| Material | Plastisol 1 | Plastisol 2 |
|---|---|---|
| SP 1000 Resin | 100 | 100 |
| Triisoamylcitrate (TIIC) | 60 | 0 |
| Tributylcitrateacetylated (TBCA) | 0 | 60 |
| CA/Zn stabilizer | 1.5 | 1.5 |
| Lubricant | 0.25 | 0.25 |

To prepare the test specimens, the above mixtures were mixed in metal cups with mechanical agitation for 5 minutes, calendered and pressed in a hydraulic press.

TABLE 11
shows the results of the test specimens.

| PLASTISSOL | 1 | 2 |
|---|---|---|
| Shore A hardness | 74 | 72 |
| Traction (kgf/mm²) | 1,586 | 1,611 |
| Elongation at break (%) | 358 | 347 |
| Mass loss in 60° C. oven after 7 days (%) | 0.004 | 0.003 |
| Volatiles at 150° C. for 5 minutes (%) | 0.4 | 0.5 |

1. A triisoamyl citrate (triisopentanol) wherein it is synthesized by the steps below:
a 2000 ml reactor is charged with 192 grams of citric acid, 400 grams of isoamyl alcohol and 1.5 grams of methane sulfonic acid;
under stirring, heat the product to 140° C. under reflux, separating the reaction water;
after 4 hours at a temperature of 130° ° C. to 140° C., 50 grams of water are collected;
place the system for distillation, raising the temperature to 160° C.;
at the end, a vacuum of 20 mmHg is applied to remove volatiles and odorous substances;
at this stage, 120 grams of recovered isoamyl alcohol were collected;
the product obtained is cooled to 80° C.;
the product is filtered through a buckner funnel and blue band filter paper.

2. A triisoamil citrate (triisopentanol) wherein it is synthesized by the steps below:
a 2000 ml reactor is charged with 192 grams of citric acid, 600 grams of isoamyl alcohol and 2.0 grams of paratoluene sulfonic acid;
under stirring, heat the product to 130° ° C. under reflux, separating the reaction water;
after 4 hours at a temperature of 120° ° C. to 130° C., 52 grams of water are collected;
place the system for distillation, raising the temperature to 160° C.;
at the end, a vacuum of 20 mmHg is applied to remove volatiles and odorous substances; at this stage, 310 grams of recovered isoamyl alcohol were collected. the product is cooled to 80° C.;
the product is filtered through a buckner funnel and blue band filter paper.

3. A triisoamil citrate (triisopentanol) wherein it is synthesized by the steps below:
a 2000 ml reactor is charged with 192 grams of citric acid, 920 grams of isoamyl alcohol and 2.0 grams of paratoluene sulfonic acid;
while stirring, heat the product to 130° C. under reflux, separating the water from the fusel oil plus the reaction water;
after 4 hours at a temperature of 120° ° C. to 130° C., 372 grams of water are collected;
place the system for distillation, raising the temperature to 160° C.;
at the end, a vacuum of 20 mmHg is applied to remove volatiles and odorous substances;
at this stage, 280 grams of recovered isoamyl alcohol were collected;
the product is cooled to 80° C.;
add 10 grams of active charcoal, stirring for 30 minutes;
the product is filtered through a buckner funnel and blue band filter paper.
